(19) United States
(12) Patent Application Publication
(10) Pub. No.: US 2011/0189238 A1
(43) Pub. Date: Aug. 4, 2011

(54) USE OF A SOLUBLE FORM OF HLA-G IN THE TREATMENT OF ABNORMAL B-LYMPHOCYTE PROLIFERATION

(75) Inventors: Nathalie Rouas-Freiss, Paris (FR); Edgardo Delfino Carosella, Paris (FR)

(73) Assignee: COMMISSARIAT A L'ENERGIE ATOMIQUE ET AUX ENE ALT, Paris (FR)

(21) Appl. No.: 13/056,845

(22) PCT Filed: Jul. 31, 2009
(86) PCT No.: PCT/FR2009/000965

§ 371 Date: Apr. 21, 2011

(30) Foreign Application Priority Data
Aug. 1, 2008 (FR) 0804404

(51) Int. Cl. A61K 39/00; C07K 14/00; A61P 35/00

(52) U.S. Cl. 424/277.1; 530/350; 424/184.1

(57) ABSTRACT

The invention relates to a novel use of the soluble forms of HLA-G in the treatment or prophylaxis of abnormal B-cell proliferation, such as liquid cancers of the B type.

The present invention relates to a novel use of the soluble forms of HLA-G in the treatment of abnormal B-cell proliferation such as liquid cancers of type B and auto-immune diseases in which the B cells are activated.

The antigens of the major histocompatibility complex (MHC) are divided into several classes, the class I antigens (HLA-A, HLA-B and HLA-C), which have 3 globular domains (α1, α2 and α3), the α3 domain being associated with 2 microglobulin, the class II antigens (HLA-DP, HLA-DQ and HLA-DR) and the class III antigens (complement).

The class I antigens comprise, apart from the aforementioned antigens, other antigens, called nonclassical class I antigens, and notably the HLA-E, HLA-F and HLA-G antigens.

The sequence of the HLA-G gene (HLA-6.0 gene) was described by GERAGHTY et al. (Proc. Natl. Acad. Sci. USA, 1987, 84, 9145-9149): it comprises 4396 base pairs and displays intron/exon organization homologous to that of the HLA-A, -B and -C genes. This gene comprises 8 exons, 7 introns and an untranslated 3′ end.

The HLA-G gene differs from the other class I genes in that the codon for translation termination, in phase, is localized at the level of the second codon of exon 6; in consequence, the cytoplasmic region of the protein encoded by this HLA-6.0 gene is shorter than that of the cytoplasmic regions of the HLA-A, -B and -C proteins. Expression of these isoforms is restricted to a few tissues such as the trophoblast (Kovats et al., 1990), the thymus (Crisa et al., 1997) and the pancreas (Cirulli et al., 2006) in nonpathological conditions.

Other research concerning this nonclassical class I antigen (ISHITANI et al., Proc. Natl. Acad. Sci. USA, 1992, 89, 3947-3951) showed that the primary transcript of the HLA-G gene can be spliced in several ways and produces at least 3 different mature mRNAs: the primary transcript of HLA-G supplies a complete copy (G1) of 1200 bp, a fragment of 900 by (G2) and a fragment of 600 by (G3). The transcript G1 does not include exon 7 and corresponds to the sequence described by ELLIS et al. (mentioned previously), i.e. it codes for a protein that comprises a signal sequence, three external domains, a transmembrane region and a cytoplasmic sequence. The mRNA G2 does not include exon 3, i.e. it codes for a protein in which the α1 and α3 domains are joined directly; the mRNA G3 contains neither exon 3 nor exon 4, i.e. it codes for a protein in which the α1 domain and the transmembrane sequence are joined directly. The splicing that prevails for obtaining the HLA-G2 antigen leads to joining of an adenine (A) (derived from the domain coding for α1) to a sequence AC (derived from the domain coding for α3), which leads to the creation of a codon AAC (asparagine) in place of the codon GAC (aspartic acid), occurring at the start of the sequence coding for the α3 domain in HLA-G1. The splicing generated for obtaining HLA-G3 does not lead to formation of a new codon in the splicing zone.

The authors of this article also analyzed the various proteins expressed: the 3 mRNAs are translated into protein in the 0.221-G cell line. They conclude, without proof, that the HLA-G molecule has a fundamental role in protection of the fetus against a maternal immune response (induction of immune tolerance).

Some of the inventors have confirmed this role: the HLA-G molecules, expressed on the surface of the trophoblasts, effectively protect the fetal cells from lysis by maternal natural killer (NK) cells (CAROSELLA E. D. et al., C.R. Acad. Sci., 1995, 318, 827-830; CAROSELLA E. D. et al., Trends Immunol. Today, 1996, 17, 9, 407-409).

Moreover, some of the inventors have shown the existence of other spliced forms of HLA-G mRNA: the HLA-G4 transcript, which does not include exon 4; the HLA-G5 transcript, which includes intron 4, between exons 4 and 5, thus causing a modification of the reading frame, during translation of this transcript and in particular the appearance of a stop codon, after amino acid 21 of intron 4; the HLA-G6 transcript, which possesses intron 4, but has lost exon 3 (KIRSZENBAUM M. et al., Proc. Natl. Acad. Sci. USA, 1994, 91, 4209-4213; European Application EP 0 677 582; KIRSZENBAUM M. et al., Human Immunol., 1995, 43, 237-241; MOREAU P. et al., Human Immunol. 1995, 43, 231-236); and the HLA-G7 transcript, which includes intron 2, thus causing a modification of the reading frame, during translation of this transcript and the appearance of a stop codon after amino acid 2 of intron 2; they also showed that these various transcripts are expressed in several types of fetal and adult human cells, notably in T and B lymphocytes (KIRSZENBAUM M. et al., Human Immunol., 1995, op. cit.; MOREAU P. et al., Human Immunol. 1995, op. cit.).

There are therefore at least 7 different HLA-G mRNAs, which potentially code for 7 isoforms of HLA-G including 4 membrane isoforms (HLA-G1, G2, G3 and G4) and 3 soluble isoforms (HLA-G5, G6 and G7).

Preliminary studies have shown that expression of HLA-G molecules on the surface of target cells obtained by transfection with vectors comprising the genomic DNA of HLA-G, potentially generating all the alternative transcripts, makes it possible to protect said target cells from the lytic activity of the NK cells of the decidual layer of the maternal endometrium (CHUMBLEY G. et al., Cell Immunol., 1994, 155, 312-322; DENIZ G. et al., J. Immunol., 1994, 152, 4255-4261).

These preliminary studies were confirmed subsequently; thus, both the membrane-bound isoforms and the soluble isoforms are immunotolerant:
they inhibit cytolysis mediated by the NK cells and the CTLs;
they inhibit the alloproliferative T response. The inhibitory action of HLA-G on the T cells is described in the literature, including that emanating from the group of M. CAROSELLA (5-13);
they induce apoptosis in the T cells and the NK CD8⁺ cells.

Thus, the HLA-G protein exerts its function locally, both when it is expressed on the surface of the cells and when it is secreted (action at a distance); it thus provides immune surveillance of the organism (Teyssier E. et al., Nat. Immunol., 1995, 14, 262-270).

Its properties of immunotolerance have also been demonstrated in vitro in many models of tumoral lines transfected with HLA-G (6, 23, 24). The HLA-G antigens play a key role in establishing and maintaining immunotolerance by inhibiting the functions of the immunocompetent cells.

These inhibitory effects are mediated by direct binding of HLA-G to specific inhibitory receptors, namely: ILT-2 (immunoglobulin-like transcript-2) (CD85j), expressed by B cells, some T cells, some NK cells, monocytes and dendritic cells, ILT-4 (CD85d), expressed by cells of myeloid lines and KIR2DL4/p49 (CD158d) (Cantoni C. et al., 1998; Rajagopalan S. et al., 1999; Naji et al., 2007), expressed by a subset CD56^{bright} of the NK cells (1-4).

These properties are shared by all of the isoforms.

Thus, HLA-G and notably the soluble isoforms such as HLA-G5:
induce apoptosis of the T CD8+ cells and of the NK cells activated by binding to the CD8 receptor and stimulation of the Fas/Fas pathway.
exert their inhibitory effects by a feedback mechanism because they inhibit the proliferative response of the alloreactive T CD4+ cells which secrete it.
have immunosuppressive properties, which they exert via their interaction with the inhibitory receptors variously expressed on the surface of the various immune cells (NK cells, T cells, B cells and antigen-presenting cells) (Carosella et al., Trends in Immunology, 2008, 29, 3, 125-132; patent application FR 2 810 047; Naji et al., 2007).
also exert their immunosuppressive activity by effects mediated by cytokines, such as IL-10 and the interferons (patent application FR 2 810 047).

The tolerogenic properties of HLA-G have beneficial effects in disorders of pregnancy, transplantation and auto-immunity and in inflammatory diseases by limiting the immune reactions, whereas they have deleterious effects in cancer and after viral infections by permitting escape of tumor cells or of cells infected by viruses.

Thus, it is now widely recognized that expression of HLA-G by tumor cells is a negative factor enabling the latter to inhibit the antitumor response through interaction of HLA-G with the inhibitory receptors of type ILT-2 expressed by the T cells and NK cells infiltrating the tumor (see the special issue of the journal Seminars in Cancer Biology on HLA-G and cancer (16)). This action of HLA-G therefore leads to tumor progression and blocking of HLA-G is accordingly now proposed as a new antitumor therapeutic approach. As an example, we may mention the works of M. Carosella's team on melanoma, showing the role of HLA-G in protecting melanomatous cells against the action of the immune system (17-20). This observation is confirmed in other types of tumors such as glioma or human renal carcinoma lines protected from an allogenic cytotoxic response by expression of HLA-G1 and HLA-G5 molecules (10, 21, 22). The many reviews on the role of HLA-G in oncology confirm these observations (2, 14, 15, 25-27).

However, all of these works relate to the action of the HLA-G molecule expressed in solid tumors.

Surprisingly, the inventors have now shown that the soluble forms of HLA-G have an antiproliferative action on the B cells of the immune system. For example, they have shown in particular the inhibitory action of the soluble forms of HLA-G on the functions of differentiation, proliferation and antibody secretion of B lymphocytes. These results have a particularly decisive impact within the scope of B-cell malignant hemopathies (lymphoma, lymphoid leukemia, myeloma, Burkitt syndrome, Hodgkin's disease etc.).

The present invention accordingly relates to the use of a soluble form of HLA-G for preparing a medicinal product for the treatment or prevention of B-cell malignant hemopathies, i.e. of pathologies in which an observed.

In other words, the present invention relates to the soluble forms of HLA-G for use as a medicinal product for the treatment or prevention of B-cell malignant hemopathies.

Such a use of HLA-G in oncology, in which the B cells are tumoral, is particularly unexpected, since it runs counter to the present concept of the role of HLA-G as a mechanism by which tumors evade immune surveillance (2, 14, 15).

Now, the inventors have found that HLA-G specifically inhibits the proliferation of tumor cells of the immune system expressing inhibitory HLA-G receptors, i.e. principally B cells. HLA-G also inhibits the proliferation, differentiation to plasmocytes and capacity to secrete antibodies of abnormally activated B cells, which means they can be used in auto-immune diseases in which the B cells are abnormally activated.

### DEFINITIONS

### B-Cell Malignant Hemopathies

The malignant hemopathies (or hematological cancers) are cancers or liquid tumors, i.e. tumors whose cells circulate in a liquid (blood or lymph), in which an abnormal B-cell proliferation is observed. Among these liquid cancers, a distinction is made between cancers of the blood (leukemia), of the bone marrow (myeloma, macroglobulinemia) or of the ganglia (lymphomas). The B-cell malignant hemopathies therefore include:
acute lymphoblastic leukemia of type B (ALL B), which affects the lymphoid progenitors (blood and bone marrow);
chronic lymphocytic leukemia (CLL), which affects the B-1 CD5 cells (blood);
pre-B leukemia, which affects the pre-B cells (blood and bone marrow);
Hodgkin's disease (lymphoma), which affects the B cells of the germinal centers;
non-Hodgkin lymphomas, such as Burkitt lymphoma or follicular lymphoma, which affect the peripheral mature memory B cells or mantle cell lymphoma which affects the peripheral naive B cells at rest;
Waldenström macroglobulinemia, which affects the IgM-secreting B cells (plasmocytes) and
multiple myeloma (or Kahler's disease).

As an example, the non-Hodgkin lymphomas are malignant tumors of the lymphatic system; there are numerous forms, which develop very differently from one another.

These lymphomas develop starting from T or B lymphocytes. B-cell tumors represent 75% of cases in western countries whereas T-cell tumors are more common in East Asia.

The incidence of non-Hodgkin lymphomas is increasing throughout the world; more than 287 000 new cases occur each year, mainly in the developed countries.

The non-Hodgkin lymphomas occur more often in the developed countries (52% of the total number of cases in the world), where their incidence has increased in the last 20 years, mainly in North America, Western Europe, Australia, Israel, Saudi Arabia. Lymphoma is also a tumoral complication observed in 5 to 10% of cases of AIDS.

### Abnormally Activated B Cells

The B cells are said to be abnormally activated when they respond to auto-antigens.

### Soluble Form of HLA-G

Said soluble form of HLA-G is selected from the group comprising HLA-G5, HLA-G6 and HLA-G7, preferably HLA-G5. These soluble forms are well known by a person skilled in the art.

The use of HLA-G in liquid cancers constitutes an alternative or complementary treatment, in combination with the treatments usually employed, as described for example in Keating M. et al. (Hematology, 2003, 153-175); Dighiero G. et al. (The Lancet, 2008, 371, 1017-1029); or Auer R. et al. (Br. J. Hematol., 2007, 139, 635-644).

The soluble forms of HLA-G, which have a mechanism of action radically different from the other anticancer products usually employed, thus offer, alone or in combination with these other products, a benefit in cases of (i) poor level of response with the other treatments, (ii) appearance of resistance to the other treatments and (iii) when the undesirable effects observed with the other treatments are too great.

HLA-G5 and, more generally, the soluble form of HLA-G, have, in liquid cancers, an antiproliferative activity and limit tumor progression.

This activity is opposite to that previously described, relating to solid cancers in which the aim is to block expression of HLA-G, to eliminate the solid tumor.

According to the invention, the soluble form of HLA-G employed is:
either in the free (or monomeric) form, which can optionally form dimers in solution,
or in multimeric form, notably in aggregated form on beads, so that the molecule of HLA-G is in the form of multimers, described as being the functionally optimal conformation of the HLA-G molecule. In fact, dimers of HLA-G have been described as displaying greatly increased affinity for the HLA-G receptors compared with the monomers.

According to the invention, the abnormal B-cell proliferation is inhibited both by the soluble form of HLA-G, purified and non-aggregated on beads, and with the aggregated forms of said soluble form of HLA-G.

The present invention also relates to a pharmaceutical composition comprising a soluble form of HLA-G and at least one pharmaceutically acceptable vehicle for use as a medicinal product for the treatment or prevention of B-cell malignant hemopathies.

According to an advantageous embodiment of said composition, said pharmaceutically acceptable vehicle is suitable for parenteral administration.

Administration can be for example intravenous, intramuscular or subcutaneous.

According to an advantageous embodiment of said composition, said pharmaceutically acceptable vehicle is suitable for administration by inhalation.

Solutions or suspensions used for subcutaneous application typically include one or more of the following compounds: a sterile diluent, such as water, for injectable preparations, a physiological saline solution, isotonic and buffered, oils, polyethylene glycols, glycerol, polypropylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, citrates or phosphates; and agents for adjusting tonicity such as sodium chloride or dextrose. The pH can be adjusted with acids or bases such as hydrochloric acid or sodium hydroxide.

These preparations can be in the form of ampules, disposable syringes or multidose bottles made of glass or plastic.

Pharmaceutical compositions suitable for injection include sterile aqueous solutions, sterile dispersions or powders for extemporaneous preparation of sterile injectable solutions or dispersions.

For intravenous administration, the preferred vehicles include physiological saline solutions, bacteriostatic water, Cremophor EL™ (BASF, Parsippany, N.J.) or PBS buffer. In all cases, the composition must be sterile and fluid. It must be stable in the conditions of preparation and storage and must comprise preservatives against the contaminating action of microorganisms such as bacteria or fungi.

As an example, the vehicle can be a solvent or a dispersion medium containing for example water, ethanol, a polyol (for example glycerol, propylene glycol or a liquid polyethylene glycol) and mixtures of these compounds.

The correct fluidity can be maintained for example by using lecithin, or by using surfactants. The action of microorganisms can be prevented by the administration of various antibacterial and antifungal agents, for example parabens, chlorobutanol, phenol, ascorbic acid and thimerosal. Said compositions can also include isotonic agents, for example sugars or polyalcohols such as mannitol, sorbitol or sodium chloride.

Prolonged action of the injectable compositions can be obtained by adding aluminum monostearate or gelatin to the formulation.

The present invention also relates to products containing a soluble form of HLA-G and an anticancer product as combined preparation for simultaneous, separate or sequential use in the treatment or prevention of cancers of B-cell malignant hemopathies.

Apart from the above arrangements, the invention further comprises other arrangements, which will become clear from the description that now follows, which refers to examples of application of the method according to the present invention, as well as to the appended drawings, in which:

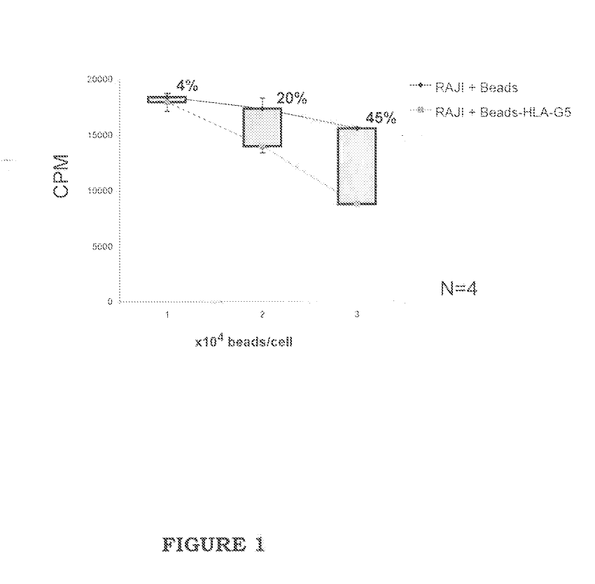

FIG. 1 illustrates inhibition of proliferation of cells of the B Raji tumoral line (ATCC accession number: CCL-86).

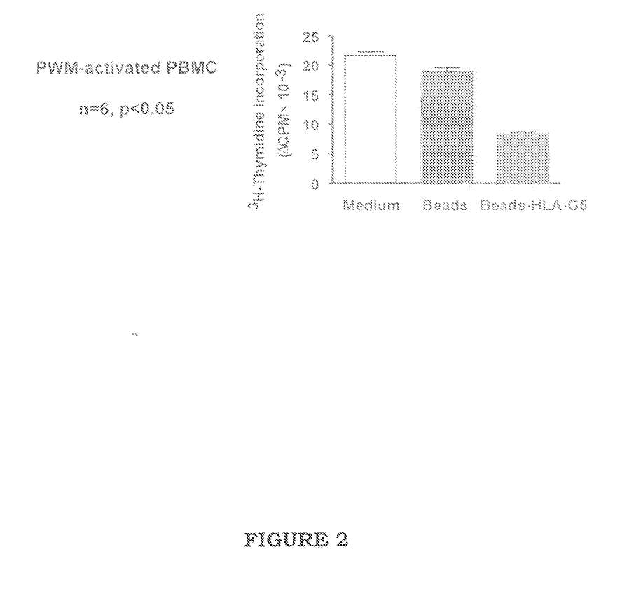

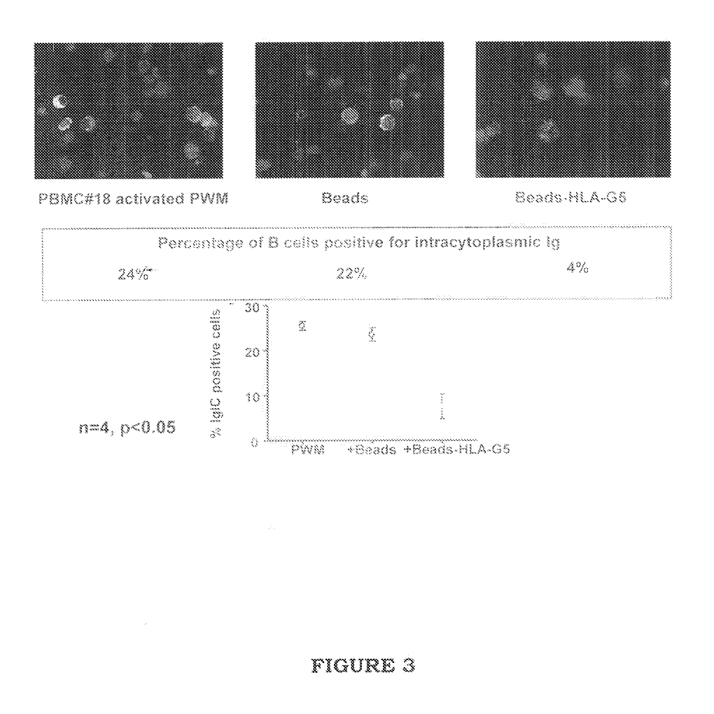

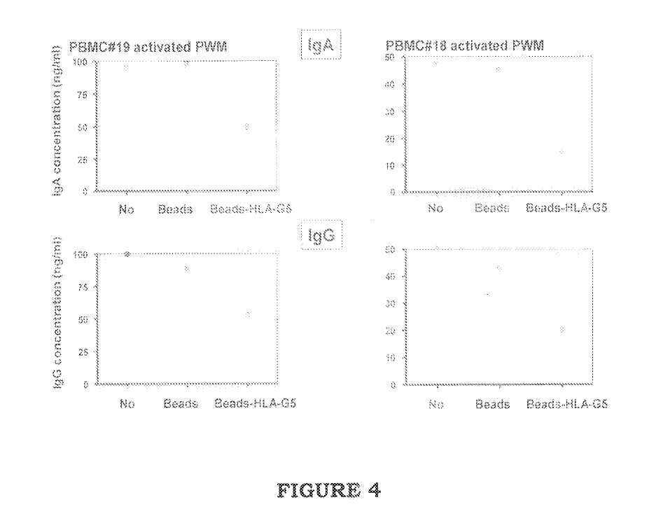

FIGS. 2, 3 and 4 illustrate the inhibitory activity of HLA-G5 on normal B cells stimulated by mitogenic agents; FIG. 2: inhibitory action of HLA-G5 on B-cell proliferation; FIG. 3: inhibitory action of HLA-G5 on differentiation of B cells to plasmocytes; FIG. 4: inhibitory action of HLA-G5 on the capacity of B cells to secrete antibodies.

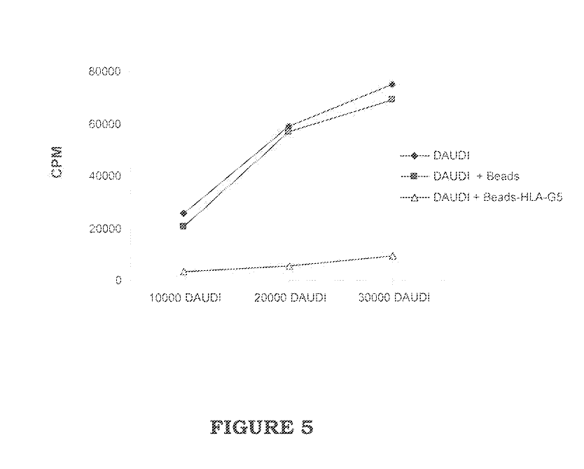

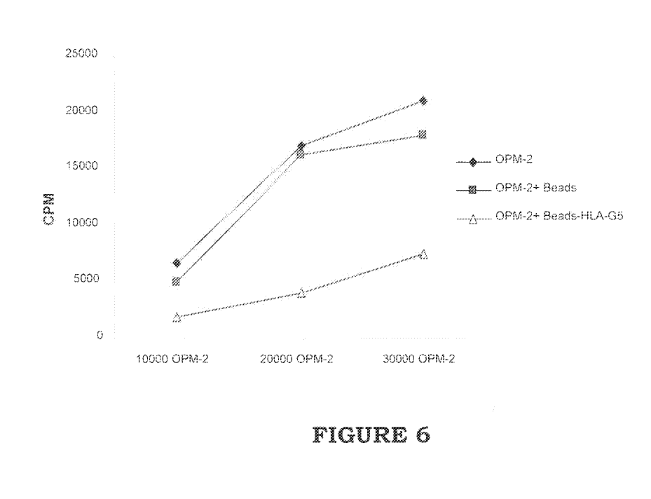

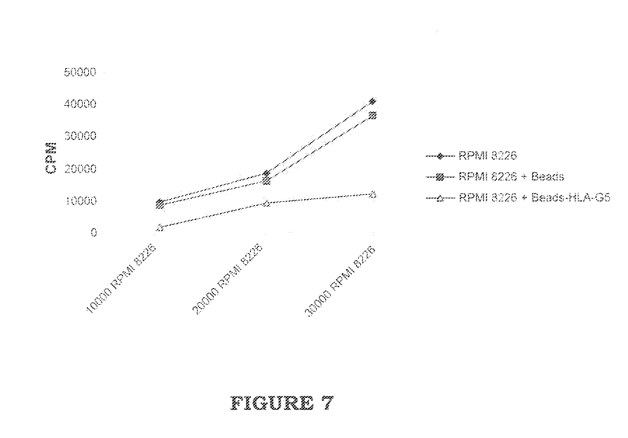

FIGS. 5, 6 and 7 illustrate inhibition of proliferation of cells of various lines; FIG. 5: Daudi tumoral line (ATCC accession number: CCL-213); FIG. 6: OPM-2 tumoral line (DSMZ accession number: ACC 50); FIG. 7: RPMI 8226 tumoral line (ATCC accession number: CCL-155).

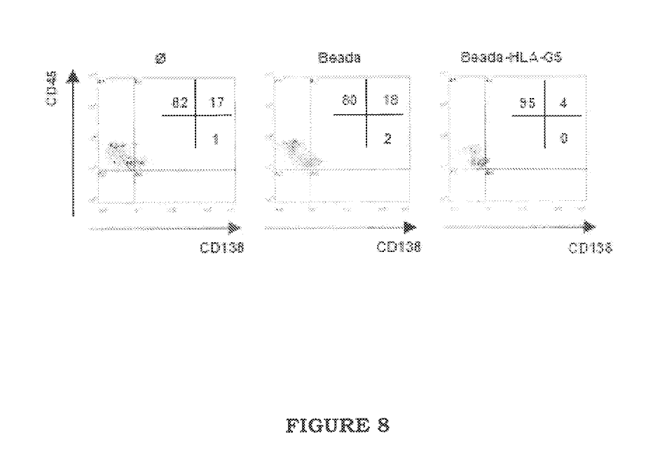

FIG. 8 illustrates inhibition of the differentiation, to malignant CD138⁺ plasmocyte cells, of CD138⁻ cells from the bone marrow of patients with multiple myeloma; the cells were sensitized in the presence (Beads-HLA-G5) or absence (0 and beads) of HLA-G5. Only one of the 3 repetitions is shown. For the cells sensitized with the culture medium without beads (“Ø”): 17 CD138⁻ cells out of 100 differentiated to CD138+ cells; for the cells sensitized with the culture medium comprising microbeads alone (“beads”): 18 CD138⁻ cells out of 100 differentiated to CD138⁺ cells; for the cells sensitized with the culture medium comprising microbeads covered with HLA-G5 (Beads-HLA-G5): 4 CD138 cells out of 100 differentiated to CD138+ cells.

However, it has to be understood that these examples are given solely for illustrating the object of the invention, and they do not in any way constitute a limitation thereof.

### EXAMPLE 1

### Materials and Methods

Cells and Cell Cultures

Two human tumoral lines of Burkitt lymphomas obtained from patients with Burkitt syndrome (Raji, ATCC accession number: CCL-86 and Daudi, ATCC accession number: CCL-213) and two myeloma cell lines (RPMI 8226, ATCC accession number: CCL-155 and OMP-2, DSMZ accession number: ACC 50) were obtained.

All the cells are cultivated on RPMI medium containing 10% of fetal calf serum, 2 mM of L-glutamine, Fungizone and gentamicin.

Peripheral blood mononuclear cells (PBMCs) are isolated from healthy volunteer donors (French Blood Establishment, St Louis Hospital, Paris, France). The PBMCs from heparinized whole blood from healthy donors are obtained by density gradient centrifugation on Ficoll-histopaque 1077 (Sigma).

CD138⁻ cell fractions were supplied by Pasteur-Cerba (Cergy, France). These cell fractions correspond to mononuclear cells from bone marrow (BM), isolated from samples obtained from patients with multiple myeloma, from which the CD138⁺ plasmocyte cells had been excluded using anti-human CD138 magnetic microbeads (Miltenyi Biotech). Informed consent had been obtained from all the patients in accordance with the Declaration of Helsinki, and the study was approved by the ethics committee.

Antibodies
W6/32: IgG2a specific to class I HLA molecules associated with 2 microglobulin (β2 m) (Sigma-Aldrich).
5A6G7: IgG1 anti-HLA-G5 and G6 (described in Le Rond et al., Eur. J. Immunol., 2004, 34, 649-660; Menier et al., Blood, 2004).

Production of HLA-G5

The protein HLA-G5 and its nucleic acid sequence are described in patent application EP 0 677 582. The production of a soluble form of an isoform of HLA-G in a baculovirus is described in detail in Example 1 of application EP 1 189 627.

Briefly, the recombinant protein HLA-G5 is produced in SF9 insect cells, cultivated on TNMFH medium containing 5% of fetal calf serum (Invitrogen), infected with a baculovirus containing the sequence coding for HLA-G5 (HLA-G5 baculovirus) or infected with the HLA-G5 baculovirus as well as with a baculovirus coding for human β2m (Appligene) and cultivated for 5 days at 27° C. in the presence of 5% CO.

The apyrogenic protein HLA-G5 is purified from the culture supernatant of infected SF9 cells by immunoaffinity chromatography with W6/32 monoclonal antibodies (Sigma-Aldrich).

Production of Recombinant HLA-G5 Adsorbed on Microbeads

Briefly, the magnetic microbeads are mono-dispersed particles with a diameter of 300 nm and are covered with goat antimouse IgG bound covalently to their surface (Bio-Adembeads goat antimouse, Ademtech).

These microbeads are incubated overnight at 4° C. with 5A6G7 monoclonal antibodies (Exbio), specific to HLA-G5.

After washing, the microbeads covered with 5A6G7 antibody are incubated with the medium containing HLA-G5 at 4° C. for 2 h.

The capture of HLA-G is verified by Western blot analysis, in the conditions described in Le Rond et al., Eur. J. Immunol., 2004, op. cit.

Tests of Cellular Proliferation

The tumoral cell lines are seeded in three wells at 10⁴ cells/100 μl of medium containing increasing amounts either of microbeads covered with HLA-G5, or of microbeads alone (negative control).

After 24 h, the cultures are pulsed with ³H-thymidine (1 μCi/well, Amersham, Biosciences).

The cells are recovered 18 h later and the incorporation of thymidine in DNA is quantified on a β counter (Wallac 1450, Pharmacia).

The peripheral blood mononuclear cells (PBMCs) (10⁵ cells/well) are activated by the mitogen pokeweed (2 μg/ml) in the absence or in the presence of HLA-G5 beads or of beads alone (2·10⁴ beads/cell).

After 5 days, the cultures are pulsed with ³H-thymidine (1 μCi/well, Amersham, Biosciences). The cells are collected 18 hours later, and the incorporation of thymidine in DNA is quantified on a β counter (Wallac 1450, Pharmacia).

Analysis of the Cell Cycle

The Raji cells are treated either with the HLA-G5 beads or with the beads alone (5·10⁴ beads/cell). After 24 h, the Raji cells are fixed in ethanol at 70% (v/v) in PBS buffer and incubated overnight at 4° C. After washing, the cells are incubated in PBS buffer containing 40 μg/ml of propidium iodide (Sigma) and 100 μg/ml of DNase without RNase A in ice, for at least 10 min.

The parameters of the cell cycle are obtained using an LSR™ flow cytometer and the software Cell Quest™ (Becton Dickinson).

The distribution of the cell cycle is determined by automatic analysis employing the software ModFit LT™ with AutoDebris™ and AutoAggregates™. The percentage of cells in each phase of the cell cycle (G0/G1, S and G2) is calculated as described in Menier C. et al. (Leukemia, 2008, 22, 578-584).

Immunofluorescent Staining of the Intracytoplasmic Immunoglobulins

The PBMCs (10⁶ cells/ml) are activated by the mitogen pokeweed (2 μg/ml) in the absence or in the presence of HLA-G5 beads or of beads alone (2·10³ beads/cell).

After 5 days, the cells are harvested from the cultures by the cytospin technique (superfrost/plus plates (Merck, Strasbourg)) and Cytospin 3⁻ (Shandon).

For staining, the cells are fixed in ethanol and incubated for 30 min with goat anti-IgG, anti-IgA and anti-IgM human antibodies labeled with FITC (fluorescein 5-isothiocyanate) or with control antibodies labeled with FITC.

The nuclei are labeled red with propidium iodide.

The plates are analyzed using a fluorescence microscope (Biorad MRC 1024). The percentage of plasmocytes positive for intracytoplasmic Ig is established by counting the cells with fluorescent cytoplasm.

ELISA

The PBMCs (10⁶ cells/ml) are activated by the mitogen pokeweed (2 μg/ml) in the absence or in the presence of HLA-G5 beads or of beads alone (2·10³ beads/cell). The human IgA and IgG secreted in the supernatants from culture of PBMCs are measured by means of the IgG ELISA and IgA ELISA quantification kits (Bethyl, Montgomery, Tex.), according to the manufacturer's instructions.

Tests of Cellular Differentiation

The CD138⁻ cell fractions obtained from bone marrow samples from patients with multiple myeloma were sensitized in vitro, for 18 to 24 h, with culture medium containing either microbeads covered with HLA-G5, or microbeads alone, or culture medium not containing microbeads. The cells were then recovered (without the microbeads) and cultivated for 21 days.

After the 3 weeks of culture, expression of CD138 on the surface of the cells among the population of CD45⁺ cells was determined by flow cytometry.

Flow Cytometry

The antibodies used for the analyses by flow cytometry were conjugated with FITC, PE (Phycoerythrin), DPE (dinitrophenyl) or PC5 (Phycoerythrin-cyanin 5) (Beckman Coulter and BD Pharmingen). Briefly, the cells were incubated for 30 min at 4° C. in 20% of human serum and then labeled with the antibodies. Isotype control was used regularly for evaluating and compensating the nonspecific signal. The cells were analyzed on an EPIC XL4 flow cytometer using the Expo32 software (Beckman Coulter).

Statistical Analysis

All the data are representative of experiments conducted at least three times. The significance was evaluated by the unpaired t test, regarding p<0.05 as significant.

### EXAMPLE 2

### Inhibition of Proliferation of B Tumor Cells (B Raji Tumoral Line)

The Raji tumoral cell line described in Example 1 was treated with beads alone or with beads covered with the soluble form HLA-G5. After 24 hours, the proliferation of the B Raji tumor cells was analyzed by incorporation of tritiated thymidine. The percentage inhibition of proliferation is shown in FIG. 1 and corresponds to the mean value obtained in four experiments.

These results are illustrated in FIG. 1 with the B Raji tumoral line, derived from a patient with Burkitt syndrome, for which there is a significant, dose-dependent decrease in proliferation after treatment with the soluble form HLA-G5. This inhibition of proliferation of B Raji tumor cells by HLA-G5 passes through a stoppage in phase G1 of the cell cycle (Table I).

TABLE I

| | G0/G1 | S | G2 |
|---|---|---|---|
| Raji | 60% | 30% | 10% |
| Raji + Beads | 60% | 30% | 10% |
| Raji + Beads-HLA-G5 | 100% | | |

The distribution during the cell cycle was defined by labeling the Raji cells with propidium iodide after 24 h of a treatment with beads-HLA-G5 or with beads alone at a rate of 50 000 beads/cell, which corresponds to 50 ng/ml of HLA-G5. The results are represented as the percentage of cells in each phase of the cycle.

### EXAMPLE 3

### Inhibition of Proliferation of B Tumor Cells (DAUDI Line, OPM-2 Line and RPMI 8226s Line)

Results similar to those of Example 2 were obtained with another Burkitt B tumoral line, the Daudi line as well as with lines derived from another type of lymphoproliferation, namely myelomas.

The Daudi, OPM-2 and RPMI 8226 cell lines described in Example 1 were treated with beads alone or with beads covered with the soluble form HLA-G5 or were not treated. After 24 hours, the proliferation of these B tumor cells was analyzed by incorporation of tritiated thymidine. The number of tumor cells introduced in the test varies from 10 000 to 30 000 cells per well. The number of beads is fixed and is 50 000 beads per cell.

The beads covered with the protein HLA-G5 inhibit the proliferation of these B tumor cells in all cases (FIGS. 5, 6 and 7).

This experiment is representative of three independent experiments.

### EXAMPLE 4

### Inhibitory Activity of HLA-G5 on Normal B Cells Stimulated by Mitogenic Agents (Pokeweed Mitogen or Pansorbine)

Inhibitory Action of HLA-G5 on B-Cell Proliferation (FIG. 2)

Peripheral blood mononuclear cells (PBMCs) isolated from a blood sample from healthy individuals were stimulated by the mitogenic agent pokeweed mitogen (PWM) in the presence (Beads-HLA-G5) or in the absence (Beads) of beads covered with the soluble form HLA-G5. After 5 days, the proliferation of the stimulated B cells was analyzed by incorporation of tritiated thymidine. These results represent the mean value obtained in 6 independent experiments. Inhibition of proliferation connected with the treatment with HLA-G5 is statistically significant. These results are shown in FIG. 2.

Inhibitory Action of HLA-G5 on Differentiation of B Cells to Plasmocytes (FIG. 3)

Peripheral blood mononuclear cells (PBMCs) isolated from a blood sample from healthy individuals were stimulated by the mitogenic agent pokeweed mitogen (PWM) in the presence (Beads-HLA-G5) or in the absence (Beads) of beads covered with the soluble form HLA-G5. After 5 days, the percentage of B cells differentiated to plasmocytes with intracytoplasmic immunoglobulins (IgIC) was determined by immunofluorescence. Inhibition of differentiation connected with the treatment with HLA-G5 is statistically significant. These results are shown in FIG. 3.

Inhibitory Action of HLA-G5 on the Capacity of B Cells to Secrete Antibodies (FIG. 4)

Peripheral blood mononuclear cells (PBMCs) isolated from a blood sample from healthy individuals were stimulated by the mitogenic agent pokeweed mitogen (PWM) in the presence (Beads-HLA-G5) or in the absence (Beads) of beads covered with the soluble form HLA-G5. After 5 days, the level of immunoglobulins IgA and IgG in the culture supernatants was measured by the immunoenzyme technique. Inhibition of the secretion of antibodies connected with the treatment with HLA-G5 is statistically significant. These results are shown in FIG. 4.

### EXAMPLE 5

### Inhibition Ex Vivo, by HLA-G5, of the Differentiation of CD138 Cells from the Bone Marrow of Patients with Multiple Myeloma to Malignant Plasma Cells CD138⁺

CD138⁺ cell tractions obtained from bone marrow from patients with multiple myeloma were sensitized for 18 h at 24 h with culture medium containing either microbeads covered with HLA-G5, or microbeads alone, or with culture medium not containing microbeads. After 3 weeks of culture without microbeads, the differentiation of CD138⁻ cells to CD138+ cells was analyzed by flow cytometry.

The results are shown in FIG. 8, from which it can be seen that HLA-G5 inhibits, at a level of 68% ((17×4)/100), the capacity of the CD138⁻ progenitor cells to differentiate to CD138⁺ cancer cells. In contrast, in the absence of HLA-G5 (Ø and beads), a significantly larger number of CD138⁻ progenitor cells differentiate to CD138⁺ malignant plasmocyte cells (respectively 17/100 and 18/100).

### BIBLIOGRAPHY

1. Carosella E D et al., Blood, 2008, 111, 4862-4870.
2. Carosella E D et al., Trends Immunol., 2008, 29, 125-132.
3. Colonna M et al., J. Exp Med., 1997, 186, 1809-1818.
4. Cosman D et al., Immunity, 1997, 7, 273-282.
5. Rouas-Freiss N et al., Proc. Natl Acad Sci USA., 1997, 94, 5249-5254.
6. Riteau Bet al., J Immunol., 2001, 166, 5018-5026.
7. Lila N et al. Proc Natl Acad Sci USA., 2001, 98, 12150-12155.
8. Ristich V et al., Eur J Immunol., 2005, 35, 1133-1142.
9. Liang Set al., Eur J Immunol., 2002, 32, 2418-2426.
10. Wiendl H et al., J Immunol., 2002, 168, 4772-4780.
11. Wiendl 1-1 et al., Brain, 2003, 126, 176-185.
12. Le Friec Get al., Hum Immunol., 2003, 64, 752-761.
13. Naji A et al., Hum Immunol., 2007, 68, 233-239.
14. Rouas-Freiss N et al., Semin Cancer Biol., 2007, 17, 413-421.
15. Urosevic M et al. Cancer Res. 2008, 68, 627-630.
16. Carosella E D et al., Semin Cancer Biol., 2007, 17, 411-412.
17. Paul P et al., Proc Natl Acad Sci USA. 1998, 95, 4510-4515.
18. Adrian Cabestre F et al., J Reprod Immunol., 1999, 43, 183-193.
19. Adrian Cabestre F et al. Semin Cancer Biol., 1999, 9, 27-36.
20. Rouas-Freiss N et al., Int J Cancer, 2005, 117, 114-122.
21. Bukur J et al., Cancer Res., 2003, 63, 4107-4111.
22. Seliger B et al., Semin Cancer Biol., 2007, 17, 444-450.
23. Menier C et al. Int J Cancer, 2002, 100, 63-70.
24. Caumartin J et al., Embo 1, 2007, 26, 1423-1433.
25. Rouas-Freiss N et al., Cancer Res., 2005, 65, 10139-10144.
26. Rouas-Freiss N et al., Semin Cancer Biol., 2003, 13, 325-336.
27. Seliger B et al., Trends Immunol., 2003, 24, 82-87.
28. Geraghty et al., Proc. Natl. Acad. Sci. USA, 1987, 84, 9145-9149.
29. Carosella E. D. et al., C.R. Acad. Sci., 1995, 318, 827-830.
30. Carosella E. D. et al, Trends Immunol. Today, 1996, 17, 9, 407-409.
31. Kovats et al., Science, 1990, 248, 220-223.
32. Crisa et al., J Exp Med., 1997, 186, 289-298.
33. Cirulli ei al., Diabetes, 2006, 55, 1214-1222.
34. Ishitani et al., Proc. Natl. Acad. Sci. USA, 1992, 89, 3947-3951.
35. Ellis et al., J. Immunol., 1990, 144, 731-735.
36. Kirszenbaum M. et al., Proc. Natl. Acad. Sci. USA, 1994, 91, 4209-4213.
37. Kirszenbaum M. et al., Human Immunol., 1995, 43, 237-241.
38. Moreau P. et al., Human Immunol. 1995, 43, 231-236.
39. Chumbley G. et al., Cell Immunol., 1994, 155, 312-322.
40. Deniz G. et al., J. Immunol., 1994, 152, 4255-4261.
41. Teyssier E. et al. Nat. Inummol., 1995, 14, 262-270.
42. Carosella et al., Adv Immunol., 2003, 81, 199-252.
43. Cantoni C. et al., Eur. J. Immunol. 1998, 28, 6, 1980-90.
44. Rajagopalan S. et al. J. Exp. Med., 1999, 189, 7, 1093-100.
45. Keating M. et al., Hematology, 2003.153-175.
46. Dighiero G. et al., The Lancet, 2008, 371, 1017-1029.
47. Auer R. et al. (Br. J. Hematol., 2007, 139, 635-644.
48. Le Road et al., Eur. J. Immunol. 2004, 34, 649-660.
49. Naji et al., Blood, 2007, 110, 12, 3936-3948.
50. Menier C. et al., Blood, 2004, 104, 10, 3153-3160.

1. A soluble form of HLA-G suitable as a medicinal product for treatment or prevention of a B-cell malignant hemopathy.

2. A pharmaceutical composition, comprising:
a soluble form of HLA-G; and
at least one pharmaceutically acceptable vehicle,
wherein the pharmaceutical composition is suitable for treatment or prevention of B-cell malignant hemopathies.

3. The soluble form of HLA-G of claim 1, selected from the group consisting of HLA-G5, HLA-G6, and HLA-G7.

4. The soluble form of HLA-G of claim 1, in free or monomeric form.

5. The soluble form of HLA-G of claim 1, in the multimeric form.

6. The composition of claim 2, wherein the soluble form of HLA-G is selected from the group consisting of HLA-G5, HLA-G6, and HLA-G7.

7. The composition of claim 2, wherein the soluble form is in free or monomeric form.

8. The composition of claim 2, wherein the soluble form is in multimeric form.

9. The composition of claim 2, wherein the pharmaceutically acceptable vehicle is suitable for parenteral administration.

10. The composition of claim 2, wherein the pharmaceutically acceptable vehicle is suitable for administration by inhalation.

11. A product, comprising:
a soluble form of HLA-G; and
an anticancer product,
as a combined preparation for simultaneous, separate, or sequential use in treating or preventing a cancer of B-cell malignant hemopathy.

12. The soluble form of HLA-G of claim 1, in the form of HLA-G5.

13. The composition of claim 2, wherein the soluble form of HLA-G is HLA-G5.

14. A method of treating a B-cell malignant hemopathy, the method comprising administering to a subject in need thereof, an effective amount of the soluble form of HLA-G of claim 1.

15. The method of claim 14, wherein the administering is parenteral.

16. The method of claim 14, wherein the administering is by inhalation.

17. A method of treating a B-cell malignant hemopathy, the method comprising administering to a subject in need thereof, an effective amount of the pharmaceutical composition of claim 2.

18. A method of treating a B-cell malignant hemopathy, the method comprising administering to a subject in need thereof, an effective amount of the product of claim 11.

19. A method of preparing the pharmaceutical composition of claim 2, the method comprising combining the soluble form of HLA-G with the at least one pharmaceutically acceptable vehicle.

20. The method of claim 19, wherein, in the combining, at least one further anticancer product is combined.
